(19) United States
(12) Patent Application Publication
(10) Pub. No.: US 2023/0076759 A1
(43) Pub. Date: Mar. 9, 2023

(54) MEDICAL CLIP AND METHOD FOR PRODUCING A MEDICAL IMPLANT

(71) Applicant: Aesculap AG, Tuttlingen (DE)

(72) Inventors: Alexander Happle, Huefingen (DE); Andreas Allerborn, Immendingen (DE); Jens Beger, Tuttlingen (DE)

(21) Appl. No.: 17/985,539

(22) Filed: Nov. 11, 2022

Related U.S. Application Data
(63) Continuation of application No. PCT/EP2021/062258, filed on May 10, 2021.

(30) Foreign Application Priority Data
May 12, 2020 (DE) 10 2020 112 781.2

(51) Int. Cl. A61B 17/122

(52) U.S. Cl. CPC A61B 17/122

(57) ABSTRACT

A medical clip, in particular in the form of an aneurysm clip, includes two clamping arms and a biasing element. A clamping arm is arranged or formed on a free end of the biasing element. The two clamping arms abut against one another in a basic position and are movable away from one another against the action of the biasing element into an open position. At least part of a surface of the clip is of colored configuration. The clip defines at least three clip regions that are spatially separate from one another. Adjacent clip regions of the at least three clip regions are of differently colored configuration.

### CROSS-REFERENCE TO RELATED APPLICATIONS

This application is a continuation under 35 U.S.C. § 365(c) of international application number PCT/EP2021/062258, filed on May 10, 2021, and claims priority to German application number 10 2020 112 781.2, filed on May 12, 2020.

The present disclosure relates to the subject matter disclosed in international application number PCT/EP2021/062258 and German application number 10 2020 112 781.2, the contents of which are incorporated herein by reference in their entireties and for all purposes.

### FIELD

The present disclosure relates to medical clips generally, and more specifically to a medical clip, in particular in the form of an aneurysm clip, which comprises two clamping arms and a biasing element, wherein in each case a clamping arm is arranged or formed on a free end of the biasing element and wherein the clamping arms in a basic position abut against one another and are movable away from one another against the action of the biasing element into an open position, wherein at least part of a surface of the clip is of colored configuration.

Furthermore, the present disclosure relates to medical clip systems generally, and more specifically to a medical clip system comprising at least two medical clips, wherein at least one clip is configured in the form of a permanent clip, the surface of which is of, in particular completely, colored configuration, and wherein at least one clip, which corresponds in shape and/or size with the permanent clip, is configured in the form of a temporary clip, the surface of which is configured at least partially corresponding in color to the permanent clip and at least partially colored for coding as a temporary clip.

Moreover, the present disclosure relates to methods for producing a medical implant generally, and more specifically to a method for producing a medical implant, in particular a medical clip or a medical screw, wherein an implant surface of the medical implant is of colored configuration.

### BACKGROUND

Clip systems of the kind described at the outset are known, in particular, in the form of aneurysm clip systems. In this case, so-called permanent clips are of completely one-colored configuration. The color hereby serves, in particular, for coding the form and/or the size of the permanent clip.

Before the final placement of such a permanent clip, which is intended to permanently remain in the body of a patient, so-called temporary clips are used during a surgical procedure, for example for treating an aneurysm. These are colored partially corresponding in color to the permanent clip that they are temporarily replacing, and partially colored as a coding indicating that this clip is a temporary clip. Such a colored design or coding of temporarily clips enables a surgeon to, for one, differentiate between sizes and/or shapes of the clips and, for another, to differentiate between temporary clips and permanent clips.

A problem with such clip systems is, in particular, that a surgeon, for example in a minimally invasive procedure, cannot always clearly recognize whether it is a temporary clip or a permanent clip. This may be due, for example, to part of the clip that is kept in one color, which codes the clip as a temporary clip, being covered by an insertion instrument. As a result, temporary and permanent clips can be mixed up in an undesired manner.

### SUMMARY

In a first aspect of the disclosure, a medical clip is provided, in particular in the form of an aneurysm clip. The medical clip comprises two clamping arms and a biasing element, wherein in each case a clamping arm is arranged or formed on a free end of the biasing element. The two clamping arms in a basic position abut against one another and are movable away from one another against the action of the biasing element into an open position, wherein at least part of a surface of the clip is of colored configuration. The clip defines at least three clip regions that are spatially separate from one another. Adjacent clip regions of the at least three clip regions are of differently colored configuration.

In a second aspect of the disclosure, a medical clip system comprises at least two medical clips, wherein, in particular, at least one clip is configured in the form of a permanent clip, the surface of which is of, in particular completely, colored configuration, and wherein at least one clip, which corresponds at least one of in shape and size with the permanent clip, is configured in the form of a temporary clip, the surface of which is configured at least partially corresponding in color to the permanent clip and at least partially colored for coding as a temporary clip. The temporary clip is configured in the form of a medical clip, which comprises two clamping arms and a biasing element, wherein in each case a clamping arm is arranged or formed on a free end of the biasing element. The two clamping arms in a basic position abut against one another and are movable away from one another against the action of the biasing element into an open position, wherein at least part of a surface of the clip is of colored configuration. The clip defines at least three clip regions that are spatially separate from one another. Adjacent clip regions of the at least three clip regions are of differently colored configuration.

In a third aspect of the disclosure, a method for producing a medical implant, in particular a medical clip or a medical screw, is proposed, wherein an implant surface of the medical implant is of colored configuration. At least three implant regions that are spatially separate from one another are defined on the implant and adjacent implant regions of the at least three implant regions are of differently colored configuration.

### BRIEF DESCRIPTION OF THE DRAWING FIGURES

The foregoing summary and the following description may be better understood in conjunction with the drawing figures, of which:

FIG. 1 shows a schematic perspective total view of an embodiment of an application instrument with an embodiment of a medical implant, held by the application instrument, in the form of a temporary implant;

FIG. 2 shows a plan view of an embodiment of a temporary implant;

FIG. 3 shows a view of the implant from FIG. 2 in the direction of arrow A;

FIG. 4 shows a plan view of a further embodiment of a temporary implant;

FIG. 5 shows a view of the clip from FIG. 4 in the direction of the arrow B;

FIG. 6 shows a plan view of a further embodiment of a temporary implant;

FIG. 7 shows a view of the implant from FIG. 6 in the direction of the arrow C;

FIG. 8 shows a plan view of a further embodiment of a temporary implant;

FIG. 9 shows a view of the implant from FIG. 8 in the direction of the arrow D;

FIG. 10 shows a plan view of a further embodiment of a temporary implant;

FIG. 11 shows a view of the implant from FIG. 10 in the direction of the arrow E;

FIG. 12 shows a plan view of a further embodiment of a temporary implant;

FIG. 13 shows a plan view of an embodiment of a permanent implant, which corresponds to the temporary implant depicted in FIG. 2;

FIG. 14 shows a view of the implant from FIG. 13 in the direction of the arrow F;

FIG. 15 shows a plan view of an embodiment of a permanent implant, which corresponds to the temporary implant depicted in FIG. 4;

FIG. 16 shows a view of the implant from FIG. 15 in the direction of the arrow G;

FIG. 17 shows a plan view of an embodiment of a permanent implant, which corresponds to the temporary implant depicted in FIG. 6;

FIG. 18 shows a view of the implant from FIG. 17 in the direction of the arrow H;

FIG. 19 shows a plan view of an embodiment of a permanent implant, which corresponds to the temporary implant depicted in FIG. 8;

FIG. 20 shows a view of the implant from FIG. 19 in the direction of the arrow I;

FIG. 21 shows a plan view of an embodiment of a permanent implant, which corresponds to the temporary implant depicted in FIG. 10;

FIG. 22 shows a view of the implant from FIG. 21 in the direction of the arrow K;

FIG. 23 shows a plan view of an embodiment of a permanent implant, which corresponds to the temporary implant depicted in FIG. 12;

FIG. 24 shows a schematic depiction of an implant before the anodic oxidation, which has two implant regions provided with a protective layer;

FIG. 25 shows a schematic view of the implant from FIG. 24 with an implant region, which was not covered with a protective layer during the anodic oxidation and on the surface of which an oxide layer is formed;

FIG. 26 shows a schematic view of the implant from FIG. 25 with the protective layer removed;

FIG. 27 shows a schematic depiction of the implant from FIG. 25 after forming an oxide layer on the implant regions, which, as depicted in FIG. 24, were covered with a protective layer before the first anodic oxidation step;

FIG. 28 shows a schematic depiction of the functioning of the interference effect of a thin oxide layer; and

FIG. 29 shows a schematic structure of an arrangement for forming an anodic oxide layer on an implant.

### DETAILED DESCRIPTION

Although the disclosure is illustrated and described herein with reference to specific embodiments, the disclosure is not intended to be limited to the details shown. Rather, various modifications may be made in the details without departing from the disclosure.

The disclosure relates to a medical clip, in particular in the form of an aneurysm clip, which comprises two clamping arms and a biasing element, wherein in each case a clamping arm is arranged or formed on a free end of the biasing element, and wherein the two clamping arms in a basic position abut against one another and are movable away from one another against the action of the biasing element into an open position, wherein at least part of a surface of the clip is of colored configuration, wherein the clip defines at least three clip regions that are spatially separate from one another and wherein adjacent clip regions of the at least three clip regions are of differently colored configuration.

Further developing a known clip, which is a concrete embodiment of a medical implant, in the described manner has the advantage, in particular, that such a medical clip, in particular, can be identified in a simple and secure manner, for example as a temporary clip. Such an identification can be ensured, in particular, with a high reliability in minimally invasive surgical procedures, under the microscope, or upon applying such a clip when same is, for example, accommodated in the mouth of an application tool and thereby partially obscured. For example, two of the three clip regions may be defined by first and second end regions of the clip. If, for example, an end or an end region of the clip forming a medical implant is grasped with an application tool and thereby obscured, the other end or the other end region of the clip is still freely visible. Therefore, a user can reliably recognize whether it is a temporary clip or a permanent clip, independently of whether the clip is partially obscured or not. Furthermore, an unambiguous differentiability of clips on an operating table or in a storage sieve basket can be achieved, in particular even in darkened rooms like, in particular, a darkened operating room. Furthermore, it should be noted that the medical clip is a medical implant, which, instead of in the form of a medical clip, in particular in the form of an aneurysm clip, may also be configured in the form of a medical screw, for example in the form of a bone screw or a pedicle screw. Such a medical implant has an implant surface, wherein at least part of the implant surface is of colored configuration, wherein at least three implant regions that are spatially separate from one another are defined on the implant and wherein adjacent implant regions of the at least three implant regions are of differently colored configuration. The disclosure is not limited to medical implants in the form of medical clips, but rather comprises any implants, in particular in the form of medical screws. Furthermore, colored configuration is to be understood to mean, in particular, that the at least three clip regions or implant regions are colored through, i.e., are made of a colored material, or only have a colored surface that leaves the viewer with a colored impression, for example by way of a coating. A substance from which the material is made may thus be differently colored than an implant surface in the at least three implant regions. In addition, adjacent is to be understood to mean that the adjacent, differently colored implant regions or clip regions adjoin one another, wherein the adjacent regions preferably do not overlap, but instead are arranged or formed next to one another without any distance or with only a small distance therebetween. An implant region or a clip region may, in particular, form any portion of the implant or the clip.

It is advantageous if at least two of the at least three clip regions are of identically colored configuration and if these at least two clip regions do not directly adjoin one another. This configuration makes it possible, in particular, to design ends that are spaced at a distance from one another on an implant in the same color. If, for example, an end is grasped and held with an application tool, a second end of the implant is then still visible for the surgeon or another user and enables a secure and reliable differentiation of such a clip, for example from a clip of one-colored configuration.

It is favorable if a first clip region of the at least two clip regions comprises free ends of the two clip arms, if a second clip region of the at least two clip regions comprises the biasing element or part thereof, and if at least one third clip region of the at least two clip regions is arranged or formed between the first clip region and the second clip region. This proposed further development makes it possible, in particular, to form a clip comprising three, four, five, or more clip regions, wherein the clip regions are differently shaped. For example, a clip having three, four, five, or more strips defined by the clip regions can thus be formed. In particular, by way of a number of the different clip regions, a probability can be minimized that all clip regions serving to identify the clip as a temporary clip are simultaneously hidden. In other words, at least one of the clip regions identifying the clip as a temporary clip thus, with a very high probability, always remains visible for a user.

The first clip region and the second clip region are preferably lighter colored than the at least one third clip region. Thus, in particular, clips of that kind can be securely identified in the surgical site, for example as temporary clips that are not intended for a permanent implantation.

Favorably at least one of the at least three clip regions is colored in one of the colors green, blue, violet, yellow, or golden. For example, the colors green, blue, and violet may be used in order to characterize the shape and/or size of the clip, yellow or golden clip regions to characterize temporary clips in order to be able to differentiate them from permanent clips, i.e. clips to be implanted permanently.

It is advantageous if either the surface of the at least one third clip region is of yellow or golden configuration, or if the surfaces of the first clip region and the second clip region are of yellow or golden configuration. In this way, it can be achieved, in particular, that at least one clip region that is yellow or golden remains visible even if the clip is grasped on an end, i.e., for example on the second clip region, and obscured by an application instrument.

In accordance with a further preferred embodiment of the disclosure, provision may be made that two adjoining clip regions of the at least three clip regions define a colored transition region of the surface and that the colored transition region is arranged or formed on a geometrically defined region of the clip. In particular, the colored transition region may be a geometrically defined region of the clip, the cross section of which is constant or conically tapers or widens on a region length, which corresponds to at least about 5%, in particular about 10%, of a total length of the clip. The colored transition region may be, in particular, much shorter than the region length in comparison to the total length of the clip. For example, adjoining clip regions may have color transitions in the form of a step function, i.e., a more or less sudden color change in spatially direct proximity. By providing the transition regions on a geometrically defined region of the clip, in particular, defined color transitions can be achieved. In particular, the geometrically defined region may be specified by a region or portion of the implant in which a cross section is constant. This may be, in particular, in the region of the clamping arms. Geometrically defined regions are, for example, also cylindrical portions or regions with a constant cross section or conical portions or regions in which a cross section increases or decreases, for example linearly. Regions of that kind may be formed, for example, in the region of a free end of the biasing element. In particular, a production of the differently colored clip regions can thus be improved, which leads to a more unambiguous coloring of the different clip regions.

It is favorable if the surface of at least one clip region of the at least three clip regions is of colored configuration by way of a coating. In particular, all clip regions may be of colored configuration by way of a coating. In particular, they may be different kinds of coatings. For example, one clip region may be formed by anodic oxidation. A second and/or third clip region may be formed, for example, by a coating with a metallic coating by means of a dip process. In particular, a plurality of layers of coatings may be arranged one on top of the other, for example in one or more clip regions. In particular, the entire clip may be of colored configuration by way of an anodic coating. A second and/or third clip region may then be provided with a metallic coating by way of an additional coating in a dip process or by way of a further anodic oxidation.

A layer thickness of the coating is preferably in a range of about 10 nm to about 500 nm. In particular, the layer thickness is in a range of about 20 nm to about 200 nm. Layer thicknesses of that kind make it possible, in particular, to use interference effects for coloration, for example if the coating is configured in the form of an oxide layer. For example, a titanium oxide layer may be formed in the described manner by anodic oxidation on a clip made of titanium. Different layer thicknesses lead, for example, to different colors. The layer thickness may be in the specified regions in the case of a dip coating, too. Thus, in particular, a secure and reliable long-lasting coating of the clip in one or more clip regions can be achieved with minimal material expenditure, which is advantageous, e.g., for gold coatings.

The clip can be formed in a simple and cost-effective manner if the coating is configured in the form of an oxide layer formed by anodic oxidation. In particular, by appropriately setting process parameters during the anodic oxidation, a color of the oxide layer can be predetermined in a desired and defined manner.

Furthermore, it may be favorable if the oxide layer forms an interference filter and if a color effect of the oxide layer depends on a layer thickness thereof. Thus, in particular, very long-lasting, stable colored coatings of clips can be formed. Desired layer thicknesses of the respective oxide layer can be specified by means of corresponding process parameters during the anodic oxidation.

The production of the clip can be further simplified if clip regions with surfaces of identically colored configuration are provided with an identical coating. Thus, in particular, two or more clip regions can be provided with identical coatings in one process step.

The coating preferably contains gold or consists of gold. Such a coating may be used, in particular, on materials to form medical clips that are not suited for anodic oxidation, for example clips that are made of cobalt chromium alloys like, in particular, an alloy commercially available under the registered trademark PHYNOX®. For example, both end regions of the clip, i.e., in particular, free ends of the clamping arms on the one hand and the biasing element on the other hand, may be coated with gold in a dip process.

The medical clip can be coated in a simple manner if the coating is configured in the form of a dip coating. For example, gold coatings can thus be achieved on non-oxidizable materials.

Furthermore, it is advantageous if at least one clip region of the at least three clip regions is uncoated. Such a clip region then has the color of the material from which the clip is made. For example, this material may have a natural oxide layer like, for example, aluminum, which is protected against corrosion by a passivating aluminum oxide layer.

The clip is preferably made of a biocompatible metal. Thus, in particular, rejection reactions of the clip after implantation can be minimized. The biocompatible metal may be, in particular, titanium, aluminum, or tantalum. Furthermore, it may be a metallic alloy.

The metallic alloy preferably contains cobalt and/or chromium and/or nickel. The stated metals may be used in appropriate combination, in particular to form implantable biocompatible implants. For example, such an alloy may be an alloy commercially available under the registered trademark PHYNOX®.

Further, the disclosure relates to a medical clip system comprising at least two medical clips, wherein, in particular, at least one clip is configured in the form of a permanent clip, the surface of which is of, in particular completely, colored configuration, and wherein at least one clip, which corresponds at least one of in shape and size with the permanent clip, is configured in the form of a temporary clip, the surface of which is configured at least partially corresponding in color to the permanent clip and at least partially colored for coding as a temporary clip, wherein the temporary clip is configured in the form of a medical clip, which comprises two clamping arms and a biasing element, wherein in each case a clamping arm is arranged or formed on a free end of the biasing element, and wherein the two clamping arms in a basic position abut against one another and are movable away from one another against the action of the biasing element into an open position, wherein at least part of a surface of the clip is of colored configuration, wherein the clip defines at least three clip regions that are spatially separate from one another and wherein adjacent clip regions of the at least three clip regions are of differently colored configuration.

A clip system further developed as proposed enables as secure differentiation of temporary clips and permanent clips. For further advantages, reference is made to the above description.

It is favorable if the color of the surface of the permanent clip forms a coding for the shape and/or size of the permanent clip. A user can thus directly recognize the shape and/or size of the permanent clips by their color or coloring. For example, a simple association with the respective shape/size of corresponding application instruments can thus also be achieved if said application instruments are provided with a corresponding coding, for example are colored completely or partially in the color of the permanent clip.

It is advantageous if each of the at least two medical clips comprises two clamping arms and a biasing element, wherein in each case a clamping arm is arranged or formed on a free end of the biasing element, and wherein the two clamping arms in a basic position abut against one another and are movable away from one another against the action of the biasing element into an open position. With clips of that kind, in particular, aneurysms or other outgrowths on hollow organs can be treated in a defined manner, for example by clamping them off.

The permanent clip and the associated temporary clip are preferably of identical configuration except for the coloration. A user can thus temporarily use a temporary clip until the permanent clip is placed and then permanently remains in the patient. A surgeon can thus first use different temporary clips in order to select the optimal clip for the respective procedure, i.e., with respect to its shape and size. After the final selection, they can then replace the temporary clip with the permanent clip. Due to the color coding of the clips configured as proposed, a surgeon can then securely differentiate whether a clip that is used is configured in the form of a temporary clip or in the form of a permanent clip.

The clip system advantageously comprises at least two permanent clips differing in size and/or shape. This enables a user to have the appropriate clip available for various indications. The clip system may, in particular, comprise five, 10, 20, or more different shapes and corresponding sizes of clips.

Further, the disclosure relates to a method for producing a medical implant, in particular a medical clip or a medical screw, wherein an implant surface of the medical implant is of colored configuration, wherein at least three implant regions that are spatially separate from one another are defined on the implant and wherein adjacent implant regions of the at least three implant regions are of differently colored configuration.

The proposed further development of the method described at the outset has, in particular, the advantages already described above in connection with preferred embodiments of medical implants in the form of medical clips. A user can reliably differentiate medical implants produced in that way, for example, from implants of one-colored configuration. Colored configuration is to be understood to mean, in particular, that the at least three implant regions are colored through, i.e., are made of a colored material, or that only the implant surface is colored, for example by coating, such that a colored impression arises in the viewer. For example, a material may be selected to form the implant, which is a different color than an implant surface in the at least three implant regions.

It is advantageous if at least two of the at least three implant regions are of identically colored configuration, wherein these at least two implant regions do not directly adjoin one another.

It is favorable if at least two of the at least three implant regions are of identically colored configuration, wherein these at least two implant regions do not directly adjoin one another. In other words, these two implant regions are spaced at a distance from one another, for example they are separated from one another by a further, differently colored implant region. Configuring two implant regions to be identically colored has the advantage, in particular, that the implant is unambiguously identifiable even when one of the two implant regions of identically colored configuration is not visible, for example because it is obscured by an instrument or tissue in the region of the surgical site. An unambiguous separation of different implant regions can thus be achieved through appropriate coloring. In addition, even in optically unfavorable conditions like, in particular, under a microscope, in a minimally invasive surgical procedure, or in darkened lighting, a user can securely identify a kind of the clip, i.e., for example temporary or permanent clips.

It is advantageous if two adjoining implant regions of the at least three implant regions define a colored transition region of the surface and if the colored transition region is arranged or formed on a geometrically defined region of the implant. In particular, the colored transition region may be arranged or formed in a geometrically defined region of the implant, the cross section of which is constant or conically tapers or widens on a region length, which corresponds to at least about 5%, in particular about 10%, of a total length of the implant. Thus, in particular, cleanly separated implant regions of different colorings can be obtained, in particular in regions in which a cross section of the implant changes or is constant. In particular, protective layers used in preferred embodiments of the method can be reliably applied in the case of such unambiguously defined geometrical regions, in order to obtain clean, sharp boundaries between adjacent clip regions of different colors.

In order to be able to optically differentiate as many different kinds of implants from one another as possible, it is advantageous if at least one of the at least three implant regions is colored in one of the colors green, blue, violet, yellow, or golden.

It is favorable if a first implant region of the at least three implant regions is defined by a first end region of the implant, if a second implant region of the at least three implant regions is defined by a second end region of the implant, and if at least one third implant region of the at least three implant regions is arranged or formed between the first implant region and the second implant region. In particular, implants with three colored clip regions can thus be formed. For example, two of the three clip regions may be of identically colored configuration. Of course, the implant may also be configured with four, five, six, or more differently colored clip regions. It is conceivable, in particular, to alternatingly color adjacent clip regions differently, i.e., for example, to provide only two colors in a total of six clip regions for identifying same. Thus, in particular, striped implants can then be formed in a simple manner.

In order to achieve, in particular, a good differentiability, even in poor light conditions, it is favorable if the first implant region and the second implant region are lighter colored than the at least one third implant region. It can thus be achieved, in particular, that even when one of the two implant regions, which are the first implant region and the second implant region, are covered and not visible to a user, the other one of these two implant regions remains visible and enables a secure differentiation of the multicolored implant from a one-colored implant.

It is advantageous if either the surface of the at least one third implant region is of yellow or golden configuration or if the surfaces of the first implant region and the second implant region are of yellow or golden configuration. For example, a yellow or golden design of the stated clip regions may be used to identify temporary implants that are only used temporarily during a surgical procedure, for example to determine an optimal size and shape of the final permanent implant to be implanted.

It is advantageous if the surface of at least one implant region, in particular all implant regions, of the at least three implant regions is provided with a colored coating. Colored coatings can be applied or formed on an implant in a simple and secure manner. In particular, they may also serve the purpose that implants made of a non-biocompatible material may be used for forming biocompatible implants, because such a coating of biocompatible materials helps to prevent a direct contact of tissue of a patient with a bioincompatible material from which the implant is produced.

The coating is preferably formed with a layer thickness in a range of about 10 nm to about 500 nm. In particular, the layer thickness may have a value in a range of about 20 nm to about 200 nm. Coatings with a layer thickness in the specified ranges can also offer, in particular, a good corrosion protection for the implant itself. Thin coatings can be formed, in particular, in a cost-effective manner. In addition, it is possible to form, for example, oxide layers with such layer thicknesses in order to obtain interference filters on the implant that predetermine a color effect of the oxide layer. In other words, coatings of different thicknesses can result in different color effects.

The coating can be configured in the form of an oxide layer in a simple manner by way of anodic oxidation. During the anodic oxidation, a layer of an oxide of the material from which the implant is produced is formed. For example, the oxide layer may be formed from titanium oxide if the implant is made of titanium.

The production of medical implants can be simplified if implant regions with surfaces of identically colored configuration are provided with an identical coating. Thus, two or more implant regions can be equipped with a surface of identically colored configuration in one process step.

Preferably, the coating is formed by gold plating. For example, this may be effected by means of a dip process. A coating of that kind has the advantage, in particular, that it is possible even with materials that are unsuited for an anodic oxidation for forming an oxide layer. For example, coatings formed by gold plating may be used with implants that are made of a cobalt chromium steels or of another material that contains cobalt and/or chromium.

Favorably at least one implant region of the at least three implant regions is left uncoated. This may be provided, in particular, when the implant material is biocompatible or already has a passivating layer. At least one method step can thus be saved, namely the method step to coat the at least one implant region for coloring same.

In accordance with a further preferred embodiment of the disclosure, provision may be made that the implant is made of a biocompatible metal and/or an anodically oxidizable metal or a metallic alloy. In particular, titanium, aluminum, or tantalum, which is comprised by the implant material, may be used as the metal. With implants of that kind, rejection reactions by the body of a patient after implanting the implant can be minimized.

It is advantageous if the implant is introduced into an electrolyte and if the implant is connected to the anode of a direct voltage source and is subjected to an anodizing voltage for the purpose of anodic oxidation. The electrolyte may be, in particular, a diluted acid. With the described approach, an oxide layer can be formed in a simple manner, namely an oxide layer corresponding to the material from which the implant is formed.

The implant can be colored with characteristic colors in a simple manner if during anodic oxidation different anodizing voltages are applied to the implant for forming different colors. For example, different implant regions can thus be colored differently by one or more implant regions being partially covered before the anodic oxidation and then the implant being subjected to a first anodizing voltage in order form a first oxide layer on the implant region that is not covered. Then, in a further method step, the clip regions already coated with an oxide layer can be covered and the implant can be subject to another, for example lower, anodizing voltage in order to provide the remaining implant regions with a differently colored oxide layer.

In accordance with a preferred embodiment of the method, provision may be made that at least one of the at least three implant regions is covered with a protective layer, that the implant is then anodically oxidized with a first anodizing voltage for forming a first oxide layer on the surface of the implant that is not covered with the protective layer, that the protective layer is removed, that the implant is then anodically oxidized with a second anodizing voltage for forming a second oxide layer on the surface of the implant, wherein the first anodizing voltage is greater than the second anodizing voltage. This approach has the advantage, in particular, that the oxide layer formed first is not altered by the second oxide layer formed in the second anodizing step, because the second anodizing voltage is lower than the first anodizing voltage. The first oxide layer thus does not change. By contrast, the second or even further implant regions that were initially covered with a protective layer are provided with the second oxide layer, which results in a different color effect due to the different anodizing voltage.

The implant can be partially covered in a simple manner if the protective layer is formed by a resin layer. For example, the implant can be dipped in resin. A dip coating can thus be achieved. When the resin is dried and cured, further regions of the implant can also optionally be covered with such a protective layer.

In order to be able to use the implant for different applications, it is favorable if it is configured in the form of a medical clip or in the form of a medical screw. In particular, the medical screw may be configured in the form of a bone screw or a pedicle screw. Of course, the use of the described method is not limited to medical implants in the form of medical clips or medical screws. In principle, the medical implant may be any kind of implant.

It is advantageous if the medical clip is configured with two clamping arms and a biasing element and if in each case a clamping arm is arranged or formed on a free end of the biasing element, wherein the two clamping arms in a basic position abut against one another and are movable away from one another against the action of the biasing element into an open position. For example, the three implant regions on the medical clip can be formed by free ends of the two clamping arms on the one hand and by the biasing element and a region formed therebetween on the other hand. Such a medical clip can be used, in particular, as an aneurysm clip for treating an aneurysm.

In order to obtain an optimal coloring of the clip, i.e., in particular, of surfaces that abut against one another in the basic position, for example clamping faces of the two clamping arms of the clip, it is favorable if the clip is opened before covering one of the at least three implant regions with the protective layer. Thus, for example, in the region of the clamping arms each clamping arm can be covered uniformly on all sides with a protective layer and then, as described above, provided with an oxide layer in a second anodizing step.

Furthermore, the use of one of the methods described above for producing a medical implant is proposed, in particular for producing a medical screw or one of the medical clips described above.

In this way, medical implants, in particular medical clips, can be colored in a desired manner so as to be differentiable by a user.

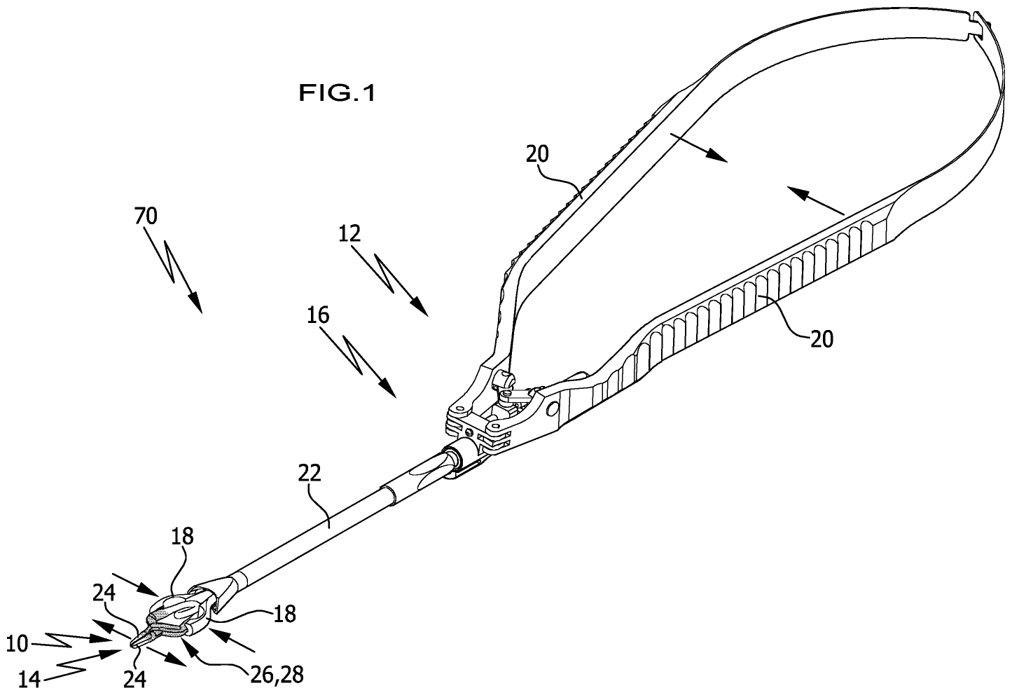

Depicted as an example in FIG. 1 is an embodiment of a medical implant 10 together with an embodiment of an application instrument 12.

The implant 10 is configured in the form of a medical clip 14, the application instrument 12 in the form of a clip applier 16.

The clip applier 16 is configured in the form of a sliding shaft instrument and comprises two tool elements 18 forming a distal end of the clip applier 16, between which a clip 14 can be accommodated. Branches 20 that are pivotable relative to one another on the proximal end of the clip applier 16 enable a movement of the shaft 22 in the distal direction toward the tool elements 18 in order to move same toward one another. Upon this movement, the surgical clip is opened, i.e., two end regions of clamping arms 24 of the clip 14 that abut against one another in a basic position, which is schematically depicted in FIG. 1, are then moved apart from one another.

The medical clip 14 depicted in FIG. 1 is configured in the form of an aneurysm clip 26 for treating aneurysms. The aneurysm clip 26 depicted in FIG. 1 is a so-called temporary clip 28. This is provided to temporarily clamp off an aneurysm during a surgical procedure. When the shape and size of the permanent clip 30 that is to be permanently implanted is determined, a surgeon can remove the temporary clip 28 and replace it with the corresponding permanent clip 30, which may permanently remain in the body of a patient.

In order to be able to differentiate temporary clips 28 and permanent clips 30 from one another, they are colored differently.

The clip 14 comprises a biasing element 32 in the form of a coil spring with 1.5 coils, the free ends of which are connected to the clamping arms 24.

Formed between the biasing element 32 and the clamping arms 24 is a connection region 34 in which the two clamping arms on the one hand and clip portions 36 connecting the free ends of the biasing elements 32 on the other hand intersect.

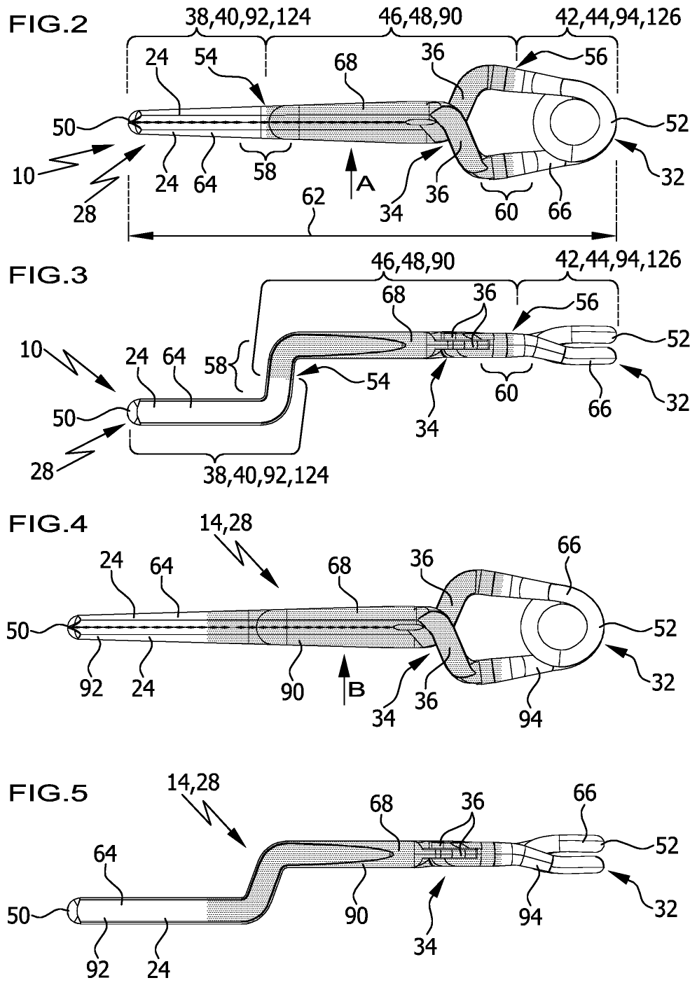

The implant 14 schematically depicted in FIG. 2 defines a first implant region 38, which defines a first clip region 40, a second implant region 42, which defines a second clip region, and a third implant region 46, which defines a third clip region 48.

The first clip region 40, which defines a first end region 124, extends commencing from a distal end 50 of the clamping arms 24 in the direction toward a proximal end 52 of the implant 10. The second clip region 44, which defines a second end region 126, extends commencing from the proximal end 52 in the distal direction. The third clip region 48 extends between the first clip region 40 and the second clip region 44.

In FIG. 2 the clip 14 is depicted in a basic position in which the clamping arms 24 abut against one another. They can be moved away from one another against the action of the biasing element 32 into an open position.

The clip regions 40, 44, and 48 are of colored configuration. In other words, the stated clip regions 40, 44, and 48 each form part of a surface of the clip 14 and are differently colored.

In the embodiment depicted in FIG. 2, the implant regions 38 and 42 are lighter colored than the third implant region 48.

Defined between the first implant region 38 and the third implant region 46 is a first transition region 54, between the second implant region 42 and the third implant region 46 a second transition region 56. The transition regions 54 and 56 are formed on a first geometrically defined region 58 and a second geometrically defined region 60, respectively. The geometrically defined regions 58 and 60 of the clip 14 are characterized in that a cross section of the clip 14 is constant or changes on a region length, which corresponds to at least about 5% of a total length 62 of the clip 14. The geometrically defined region 58 on the clamping arms 24 is characterized by a constant cross section. The geometrically defined region 60, which directly adjoins the biasing element 32, has a cross section that conically widens from the biasing element 32 in the direction toward the connection region 34 and transitions into a cylindrical portion of the clip 10.

The three implant regions 38, 42, and 46 are each provided with a coating 64, 66, and 68. The production of these coatings 64, 66, and 68 as well as their structure are explained in more detail in the following.

FIGS. 4 to 12 show different embodiments of surgical clips 14. These differ exclusively in a shape of their clamping arms 24. Therefore, all embodiments are provided with the same reference numerals.

The embodiment of FIGS. 2 and 3 shows double angled clamping arms 24. A further embodiment of a clip 14 with double angled clamping arms, but which are slightly longer compared to the embodiment of FIGS. 2 and 3, is schematically depicted in FIGS. 4 and 5.

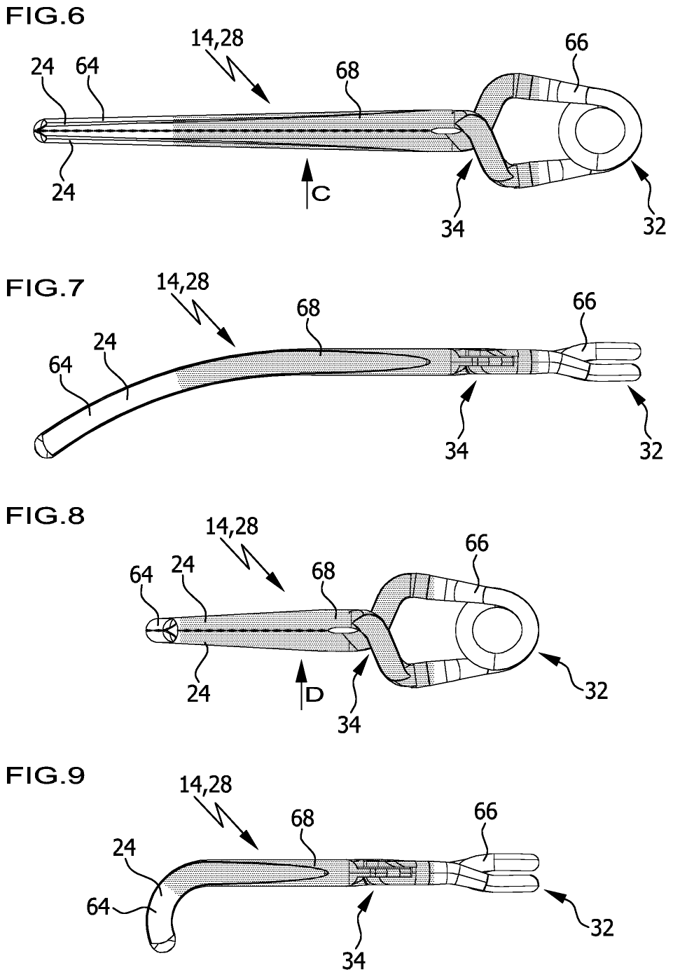

In the embodiment of the clip 14 from FIGS. 6 and 7, the clamping arms 24 are elongately arced with a relatively large radius of curvature.

In the embodiment of the clip 14 from FIGS. 8 and 9, the clamping arms 24 are curvedly arced, but with a significantly smaller radius of curvature in comparison to the embodiment of FIGS. 6 and 7.

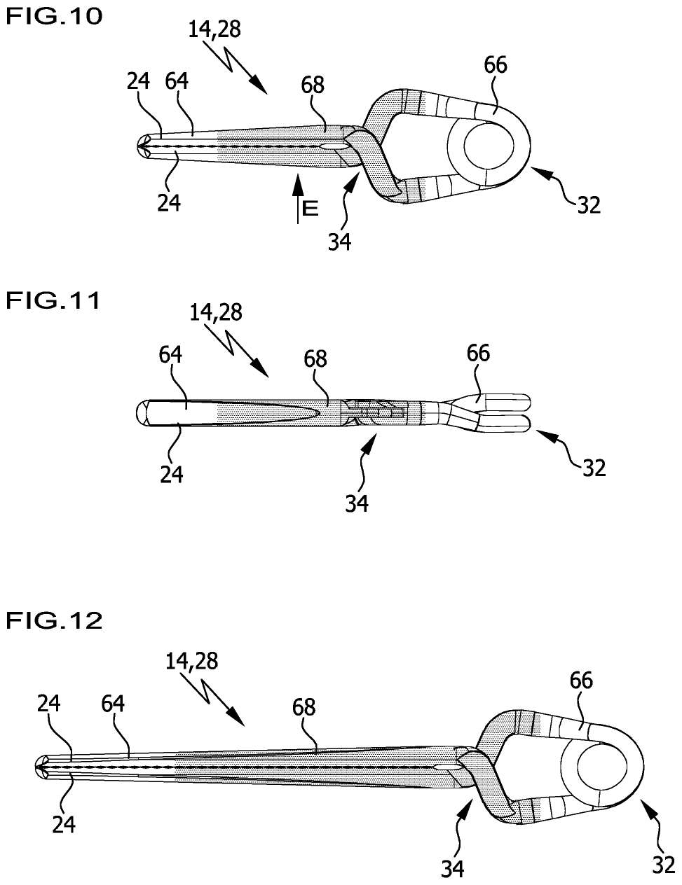

The embodiment of the clip of FIGS. 10 and 11 shows clamping arms 24 extending rectilinearly.

The clamping arms of the embodiment of the clip 14 depicted in FIG. 12 also extend rectilinearly, but are significantly longer in comparison to the embodiment of FIGS. 10 and 11.

FIGS. 1 to 12 show temporary clips 28.

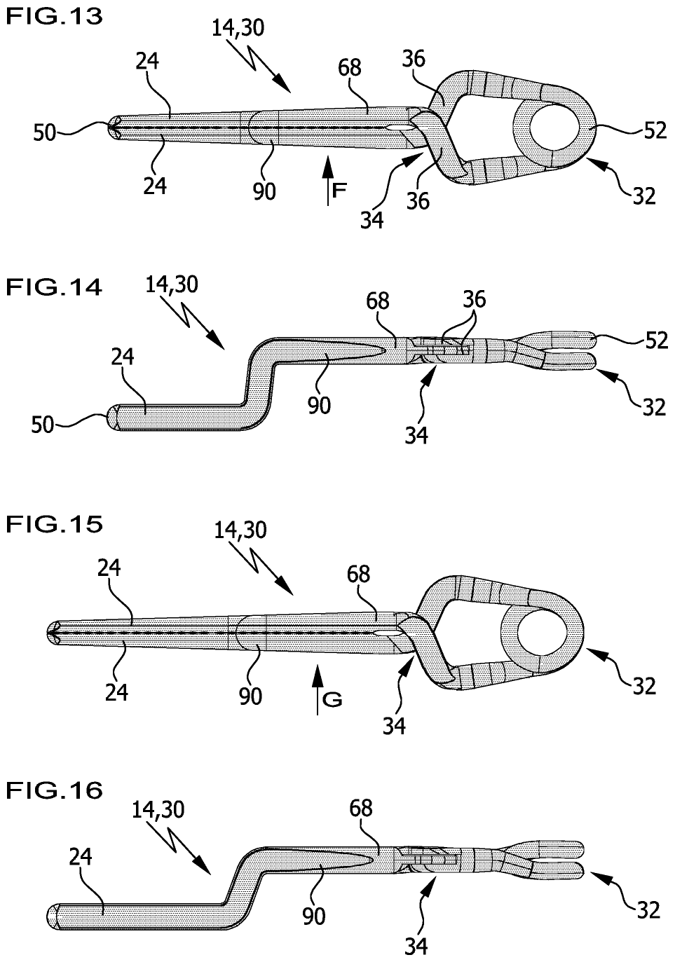

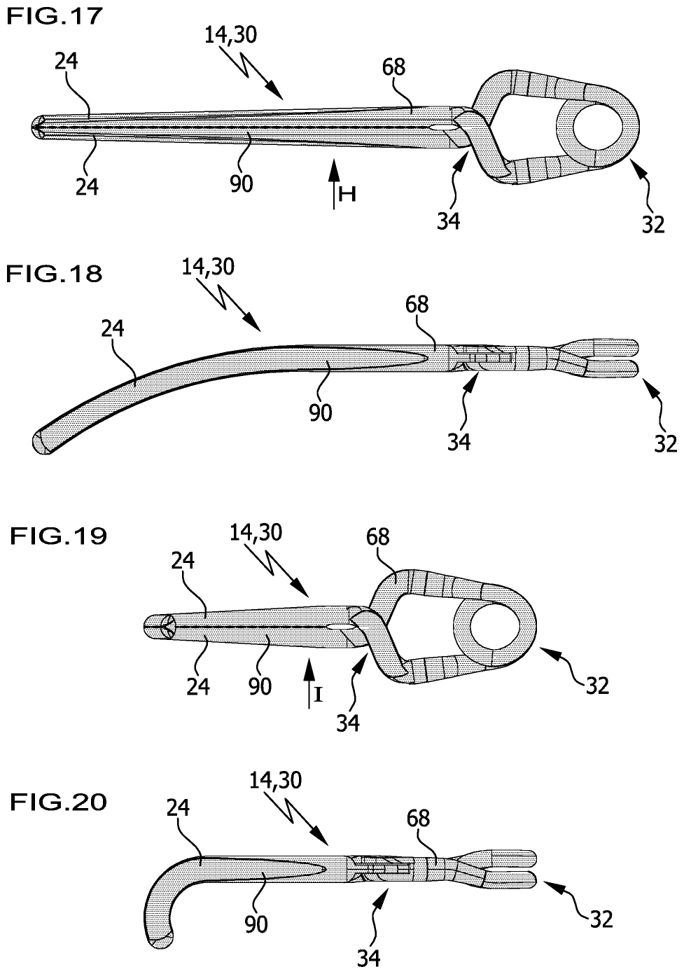

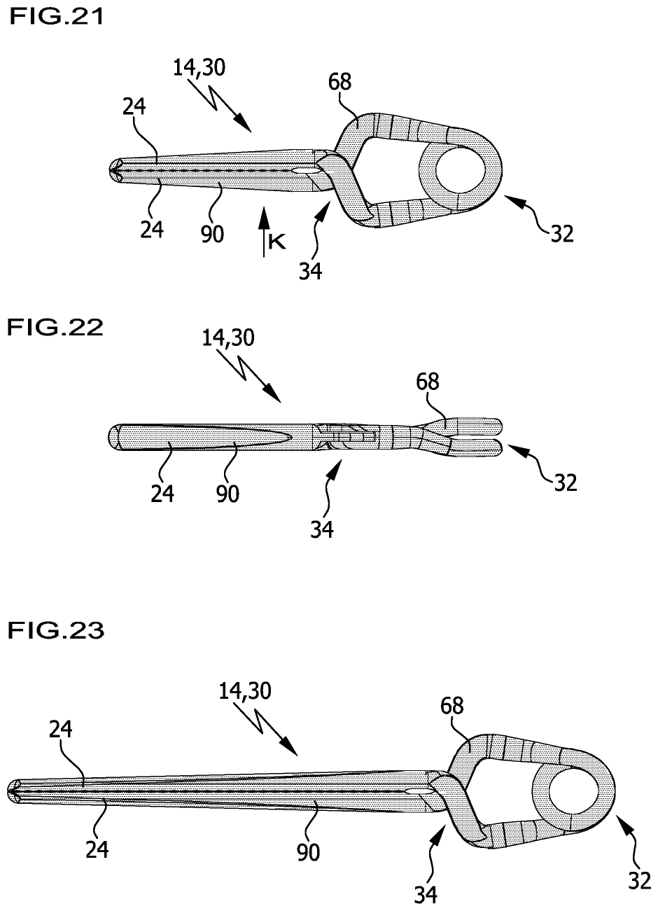

The embodiments of permanent clips 30 depicted in FIGS. 13 to 23 correspond in shape and size with the respectively associated embodiments of the temporary clips 28 of FIGS. 2 to 12. However, they differ from the temporary clips 28 in their coloring. All embodiments of the permanent clips that are schematically depicted in FIGS. 13 to 23 are of one-colored configuration. They are colored in one color that corresponds to the coloring of the third implant region 46 of the temporary clips 28. As a result, a simple association of temporary clips 28 and corresponding permanent clips 30 is possible.

The differently shaped and sized embodiments of permanent clips of FIGS. 13 and 14, 15 and 16, 17 and 18, 19 and 20, 21 and 22 and 23 optionally differ in their coloring. For example, one embodiment may be green, another embodiment blue, a further embodiment yellow, a further embodiment red, and a further embodiment violet. Correspondingly, the third implant regions 16 or the third clip regions 48 of the temporary clips 28 are then colored corresponding to the permanent clips 30 of identical design and size.

In order to be able to securely differentiate the temporary clips 28 from the permanent clips 30, in the case of the temporary clips 28, the first clip regions 40 and the second clip regions 44 are differently colored than the third clip regions 48.

In the embodiments depicted in FIGS. 1 to 12, the first and second clip regions 40, 44 are colored yellow or golden. They are thus significantly lighter colored than the third implant regions 46 or the third clip regions 48. A user can then still securely differentiate the temporary clips 28 from permanent clips 30 even when the temporary clips 28 are held, for example, with the clip applier 16 schematically depicted in FIG. 1 and the biasing elements 32 are only poorly visible or not visible at all.

However, the first implant regions 38 of the temporary clips 28 are easily visible, in particular even under a microscope, such that a user can immediately recognize, in particular also in a minimally invasive surgical procedure, whether they are handling a temporary clip 28 or a permanent clip 30.

Both the permanent clips 30 and the clip regions 40, 44 and 48 of the described embodiments of temporary clips 28 depicted in the Figures are formed by anodic oxidation in the depicted embodiments.

For this purpose, first the respective clip 28 or 30, made of a biocompatible metal, namely titanium, aluminum or tantalum being used in the embodiments, is configured in the desired form, which is depicted as an example in FIGS. 2 to 23.

In alternative embodiments, the respective clip 28, 30 is made of a metallic alloy that contains cobalt and/or chromium and/or nickel.

In one embodiment, the clips are made of an alloy commercially available under the registered trademark PHYNOX®.

With materials that are anodically oxidizable like, e.g., titanium, aluminum, and tantalum, a coating device 72 as schematically depicted in FIG. 29 may be used. The coating device 72 comprises a direct voltage source 74 and an electrolysis cell 76, which is filled with an electrolyte 78 in the form of a diluted acid.

The implant 10 to be coated is introduced into the electrolyte 78, such that it is completely surrounded by the electrolyte 78.

The implant 10 is connected to the positive pole 80, i.e., the anode, of the direct voltage source 74 and an anodizing voltage 82 is set by means of a potentiometer 84. In the embodiment of the coating device 72 depicted in FIG. 29, a container 86 accommodating the electrolyte 78, which is electrically conductively connected to the minus pole 88, i.e., the cathode, of the direct voltage source 74, serves as a counter electrode.

By means of the coating device 72, the embodiments of permanent clips 30 depicted in FIGS. 13 to 23 are provided with an oxide layer in one single anodizing step. For this purpose, the anodizing voltage 82 is specified with the potentiometer 84 depending on the desired coloring. The permanent clips are completely dipped into the electrolyte 78 as described and electrically conductively connected to the anode of the direct voltage source 74. In dependence on the anodizing voltage 82, the oxide layers are then colored in a desired color, for example any spectral color like, in particular, green, blue, violet, red, or yellow.

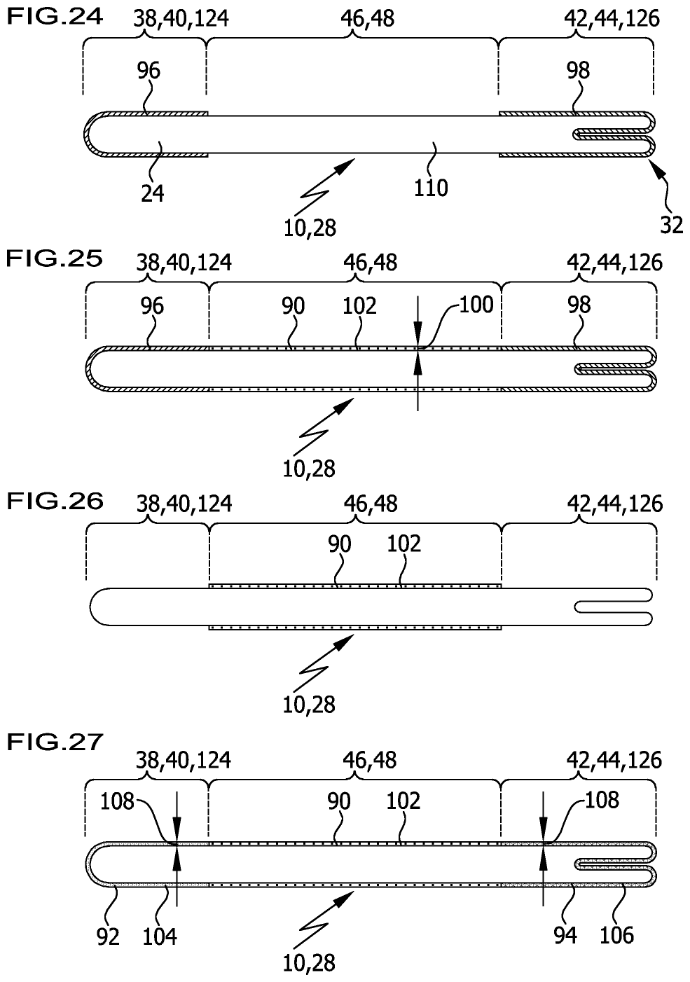

For forming a colored coating 90 of the implant regions 38, 42, and 46 of the embodiments of temporary clips 28 depicted in FIGS. 2 to 12, one proceeds as subsequently discussed in more detail in connection with FIGS. 24 to 27.

In order to provide the third implant region 46 with the coating 90 in a first step, the first and second implant regions 38 and 40 are first provided with a respective protective coating 96 and 98, which completely covers the respective implant regions 38, 42. Then only the third implant region 46 remains uncoated.

When the implant 10 that is partially covered with the protective layers 96 and 98 in that way is introduced into the electrolysis cell 76 in the manner described above, the coating 90 can be formed by applying a first anodizing voltage U₁.

The coating is formed with a thickness 100. It completely covers the third implant region 46. A configuration of such a coating 90 in the form of such an oxide layer 102 is not possible in the second and third implant regions 42, 46 because they are covered by the protective layers 96 and 98 and are thereby passivated.

In order to also provide the implant regions 42 and 46 with a respective coating 92 and 94, first the protective layers 96 and 98 are removed. The implant 10 prepared in this way is schematically depicted in FIG. 26. Only the third implant region 46 is provided with the coating 90.

If the implant 10 prepared in this way is now in turn dipped into the electrolysis cell 76 and connected to the plus pole 80 of the direct voltage source 74 and a second anodizing voltage U₂ is applied that is smaller than the first anodizing voltage U₁, a respective oxide layer 104 and 106 also forms on the implant regions 38 and 42. Due to the lower anodizing voltage U₂, the oxide layer 102 that is already formed is not further altered by this second oxidation step.

The oxide layers 104 and 106 each have a thickness 108.

All embodiments of temporary clips 28 depicted in FIGS. 2 to 12 and described above can be formed in the described manner with three differently colored clip regions 40, 44, and 48. As already mentioned, an association of corresponding permanent clips 30 and temporary clips 28 is brought about by the identical configuration of at least one of the three implant regions 38, 42, and 46 with a coating 90 that corresponds to a coating 90 of the permanent clips 30.

The coatings 90, 92, and 94 have a thickness 100 or 108, which is in a range of about 10 nm to about 500 nm. In embodiments, it is in a range of about 20 nm to about 200 nm.

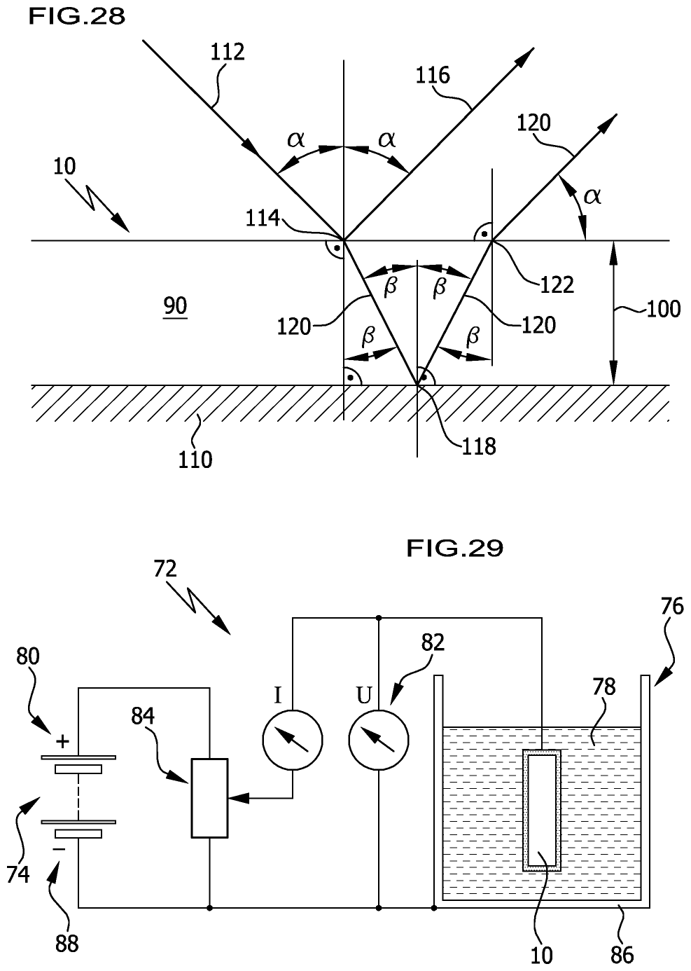

The coloring of the oxide layers 102 to 106 formed as described is discussed in the following in connection with FIG. 28.

Schematically depicted in FIG. 28 is the oxide layer 90, which is formed on the anodically oxidizable material 110 from which the implant 10 is made.

Incident light 112 is partially reflected at the point of incidence 114 on the oxide layer 90 and is deflected as a wavefront 116 at an angle of reflection α, which corresponds to an angle of incidence α of the light 112 on the oxide layer.

A portion of the light 112 penetrates into the coating 90 and is broken toward the perpendicular in this optically denser medium. At the point 118, i.e., on the boundary face between the coating 90 and the material 110, a total reflection of the wavefront 120 takes place on the optically denser medium, which then exits the coating 90 and the exit point 122. The two wavefronts 116 and 120 overlap interferometrically and result in a different coloring of the coating 90 in dependence on the kind and thickness 100 of the coating 90.

If the implants 10 are not made of an oxidizable material, the coatings may also be realized in a dip process. For example, the first and second implant regions 38 and 42 are provided with a gold coating by dip coating in embodiments of the implants 10. The third implant regions 46 are either not coated or before the coating of the first and second implant regions 38 and 42 are provided with another coating that differs in color from a golden coating. In this case then, the permanent clips 30 that are schematically depicted in FIGS. 13 to 23 are made from the same material as the temporary clips 28 and in turn are provided with a coating 90 that corresponds to the coating 90 of the third implant regions 46 of the temporary clips 28.

In further embodiments, the coating process described in connection with FIGS. 24 to 27 is implemented vice versa. In this case, first the third implant region 46 was covered with the protective layer 96 or 98 and the first and second implant regions 38 and 42, which define first and second end regions 124, 126, were provided with the respective coating 92 and 94 by anodic oxidation. In the next step, the protective layer 96 on the third implant region 46 was removed and the implant 10 was anodically oxidized again, but with a lower anodizing voltage U₂.

The described medical clip system 70 comprises at least two medical clips 14, of which at least one clip 14 is configured in the form of a permanent clip 30 and at least one clip 14 is configured in the form of a temporary clip 28. The shape of the temporary clip 28 corresponds to the shape and size of the permanent clip 30. The association of the temporary clips 28 with the permanent clips 30 is achieved, as described, by the temporary clip 28 comprising a clip region 48 that is colored corresponding in color to the permanent dip 30.

Embodiments of medical implants 10 in the form of clips 14 are described in connection with FIGS. 1 to 23.

The colored design of surfaces of the implants 10 is also used to correspondingly identify embodiments of temporary and permanent implants that are configured in the form of screws. An implant region, for example the third implant region 46, may be used in these screws to identify and code, in particular, a thickness, a length, or the respective screw type.

An association of colors with the respective property of the implant 10 is freely selectable in the production of the implant 10.

1. A medical clip comprising two clamping arms and a biasing element, wherein in each case a clamping arm is arranged or formed on a free end of the biasing element, and wherein the two clamping arms in a basic position abut against one another and are movable away from one another against the action of the biasing element into an open position, wherein at least part of a surface of the clip is of colored configuration, wherein the clip defines at least three clip regions that are spatially separate from one another and wherein adjacent clip regions of the at least three clip regions are of differently colored configuration.

2. The medical clip according to claim 1, wherein at least two of the at least three clip regions are of identically colored configuration and wherein these at least two clip regions do not directly adjoin one another.

3. The medical clip according to claim 1, wherein a first clip region of the at least three clip regions comprises free ends of the two clamping arms, wherein a second clip region of the at least three clip regions comprises the biasing element or part thereof, and wherein at least one third clip region of the at least three clip regions is arranged or formed between the first clip region and the second clip region.

4. The medical clip according to claim 1, wherein at least one of the at least three clip regions is colored in one of the colors green, blue, violet, yellow, or golden.

5. The medical clip according to claim 1, wherein two adjoining clip regions of the at least three clip regions define a colored transition region of the surface and wherein the colored transition region is arranged or formed on a geometrically defined region of the clip, the cross section of the geometrically defined region being constant or conically tapers or widens on a region length, which corresponds to at least about 5% of a total length of the clip.

6. The medical clip according to claim 1, wherein the surface of at least one clip region of the at least three clip regions is of colored configuration by way of a coating.

7. The medical clip according to claim 6, wherein at least one of:
a) a layer thickness of the coating is in a range of about 10 nm to about 500 nm; and
b) the coating is configured as an oxide layer (102, 104, 106) formed by anodic oxidation; and
c) clip regions with surfaces of identically colored configuration are provided with an identical coating; and
d) the coating contains gold or consists of gold.

8. The medical clip according to claim 1, wherein at least one of:
a) at least one clip region of the at least three clip regions is uncoated; and
b) the clip is made of a biocompatible metal.

9. A medical clip system comprising at least two clips, a first clip of the at least two clips configured as a permanent clip, the surface of which is of colored configuration, and wherein a second clip of the at least two clips, which corresponds at least one of in shape and size with the permanent clip, is configured as a temporary clip, the surface of which is configured at least partially corresponding in color to the permanent clip and at least partially colored for coding as the temporary clip, wherein the temporary clip is configured in the form of a medical clip, which comprises two clamping arms and a biasing element, wherein in each case a clamping arm is arranged or formed on a free end of the biasing element, and wherein the two clamping arms in a basic position abut against one another and are movable away from one another against the action of the biasing element into an open position, wherein at least part of a surface of the clip is of colored configuration, wherein the clip defines at least three clip regions that are spatially separate from one another and wherein adjacent clip regions of the at least three clip regions are of differently colored configuration.

10. The medical clip system according to claim 9, wherein at least one of:
a) the color of the surface of the permanent clip forms a coding for at least one of the shape and size of the permanent clip; and
b) each of the at least two medical clips comprises two clamping arms and a biasing element, wherein in each case a clamping arm is arranged or formed on a free end of the biasing element, and wherein the two clamping arms in a basic position abut against one another and are movable away from one another against the action of the biasing element into an open position and
c) the permanent clip and the temporary clip are of identical configuration except for the coloring and
d) the clip system comprises at least two permanent clips differing in at least one of size and shape.

11. A method for producing a medical implant, wherein an implant surface of the medical implant is of colored configuration, wherein at least three implant regions that are spatially separate from one another are defined on the implant and wherein adjacent implant regions of the at least three implant regions are of differently colored configuration.

12. The method according to claim 11, wherein at least one of:
a) at least two of the at least three implant regions are of identically colored configuration, wherein these at least two implant regions do not directly adjoin one another; and
b) two adjoining implant regions of the at least three implant regions define a colored transition region of the surface and wherein the colored transition region is arranged or formed on a geometrically defined region of the implant, the cross section of the geometrically defined region being constant or conically tapers or widens on a region length, which corresponds to at least about 5% of a total length of the implant.

13. The method according to claim 11, wherein at least one of:
a) at least one of the at least three implant regions is colored in one of the colors green, blue, violet, yellow, or golden; and
b) a first implant region of the at least three implant regions is defined by a first end region (124) of the implant, wherein a second implant region of the at least three implant regions is defined by a second end region (126) of the implant, and wherein at least one third implant region of the at least three implant regions is arranged or formed between the first implant region and the second implant region.

14. The method according to claim 13, wherein at least one of:
a) the first implant region and the second implant region are lighter colored than the at least one third implant region; and
b) either the surface of the at least one third implant region is of yellow or golden configuration, or wherein the surfaces of the first implant region and the second clip region are of yellow or golden configuration.

15. The method according to claim 11, wherein at least one of:
a) the surface of at least one implant region of the at least three implant regions is provided with a colored coating; and
b) at least one implant region of the at least three implant regions are left uncoated, and
c) the implant is made of at least one of a biocompatible metal and an anodically oxidizable metal or a metallic alloy, wherein the metal is or contains titanium, aluminum, or tantalum.

16. The method according to claim 15, wherein at least one of:
a) the coating is configured with a layer thickness in a range of about 10 nm to about 500 nm; and
b) the coating is configured in the form of an oxide layer (102, 104, 106) formed by anodic oxidation; and
c) implant regions with surfaces of identically colored configuration are provided with an identical coating; and
d) the coating is formed by gold plating.

17. The method according to claim 16, wherein the implant is introduced into an electrolyte, and wherein the implant is connected to the anode of a direct voltage source and is subjected to an anodizing voltage for anodic oxidization.

18. The method according to claim 17, wherein at least one of:
a) different anodizing voltages are applied to the implant during the anodic oxidation for forming different colors; and
b) at least one of the at least three implant regions is covered with a protective layer, wherein the implant is then anodically oxidized with a first anodizing voltage for forming a first oxide layer on the surface of the implant that is not covered with the protective layer, wherein the protective layer is removed, wherein the implant is then anodically oxidized with a second anodizing voltage for forming a second oxide layer on the surface of the implant, wherein the first anodizing voltage is greater than the second anodizing voltage.

19. The method according to claim 11, wherein the medical implant is configured in the form of a medical clip configured with two clamping arms and a biasing element and wherein in each case a clamping arm is arranged or formed on a free end of the biasing element, wherein the two clamping arms in a basic position abut against one another and are movable away from one another against the action of the biasing element into an open position.

20. A method for producing a medical implant having an implant surface with a colored configuration and at least three implant regions that are spatially separate from one another, the at least three implant regions comprising adjacent implant regions that are of differently colored configuration, the method comprising the steps of: producing two clamping arms and a biasing element, each of the two clamping arms being arranged or formed on a free end of the biasing element, wherein the two clamping arms abut against one another in a basic position and are movable away from one another against the action of the biasing element into an open position, wherein at least part of a surface of the clip is of colored configuration, wherein the clip defines at least three clip regions that are spatially separate from one another and wherein adjacent clip regions of the at least three clip regions are of differently colored configuration.
